(19) United States
(12) Patent Application Publication
(10) Pub. No.: US 2014/0309508 A1
(43) Pub. Date: Oct. 16, 2014

(54) DIAGNOSTIC MODULE FOR DIAGNOSING DISEASE AND DISEASE DIAGNOSIS APPARATUS HAVING THE SAME

(71) Applicant: Industry-Academic Cooperation Foundation, Chosun University, Gwangju (KR)

(72) Inventors: Youn-Tae KIM, Daejeon (KR); Ji-Hwan Lee, Jeollanam-do (KR); Jae-Hyo Jung, Gwangju (KR); Ji-Hoon Lee, Gwangju (KR)

(21) Appl. No.: 13/912,981

(22) Filed: Jun. 7, 2013

(30) Foreign Application Priority Data
Apr. 11, 2013 (KR) 10-2013-0039867

(51) Int. Cl. A61B 5/15; A61B 5/1477

(52) U.S. Cl. CPC A61B 5/1411; A61B 5/1477
USPC 600/345; 600/309

(57) ABSTRACT

There are provided a diagnostic module for diagnosing a disease and a disease diagnosis apparatus including the same. The disease diagnosis apparatus includes a patch including one or more diagnostic module attachable-detachable recesses, one or more diagnostic modules detachably attached to the diagnostic module attachable-detachable recesses to collect and analyze blood, and a processor processing analysis results.

### PRIORITY

This application claims the priority of Korean Patent Application No. 2013-39867 filed on Apr. 11, 2013, in the Korean Intellectual Property Office, the disclosure of which is incorporated herein by reference.

### BACKGROUND OF THE INVENTION

1. Field of the Invention

The present invention relates to a diagnostic module for diagnosing a disease and a disease diagnosis apparatus including the same. The present invention is derived from research conducted as part of the National Research Foundation of the Korean-Public Welfare & Security R&D Program supported by the Ministry of Education and Science Technology [Project Management No.: 2012-0006526, Project Title: Development of Body-Mounted Sensor Module for Preventing Acute Circulatory System Functional Disorder and Clinical Performance Evaluation].

2. Description of the Related Art

As the point-of-care testing (POCT) market has expanded rapidly, numerous disease diagnosis apparatuses for diagnosing diseases in domestic settings have been developed. In general, disease diagnosis apparatuses for diagnosing diseases in domestic settings are too costly and have may inconvenience in diagnosing diseases in daily lives.

### SUMMARY OF THE INVENTION

An aspect of the present invention provides a diagnostic module for simply diagnosing diseases at low cost in domestic settings and a disease diagnosis apparatus including the same.

According to an aspect of the present invention, there is provided a disease diagnosis apparatus including: a patch including one or more diagnostic module attachable-detachable recesses; one or more diagnostic modules detachably attached to the diagnostic module attachable-detachable recesses to collect and analyze blood; and a processor processing analysis results.

The patch may further include: attachable-detachable buttons corresponding to the diagnostic module attachable-detachable recesses, respectively, wherein each of the diagnostic modules may be separated from the diagnostic module attachable-detachable recesses through an attachable-detachable button pressed by a user.

Each of the diagnostic modules may include: a blood collecting unit collecting blood using a microneedle, based on pressure applied to a corresponding diagnostic module; a sensor unit detecting a current signal generated by an oxidation-reduction reaction between antibodies reacting to a corresponding cardiac marker, among cardiac markers, and antigens contained in the collected blood by using a three-dimensional (3D) electrochemical sensor; and a high-sensitivity signal sensing circuit amplifying and filtering the detected current signal and providing the amplified and filtered current signal to the processor, wherein the blood collecting unit, the sensor unit, and the high-sensitivity signal sensing circuit are integrated into a single module.

According to another aspect of the present invention, there is provided a diagnostic module. The diagnostic module includes a blood collecting unit collecting blood using a microneedle, based on pressure applied to a corresponding diagnostic module; a sensor unit detecting a current signal generated by an oxidation-reduction reaction between antibodies reacting to a corresponding cardiac marker, among cardiac markers, and antigens contained in the collected blood by using a three-dimensional (3D) electrochemical sensor; and a high-sensitivity signal sensing circuit amplifying and filtering the detected current signal, wherein the blood collecting unit, the sensor unit, and the high-sensitivity signal sensing circuit are integrated into a single module.

The foregoing technical solutions do not fully enumerate all of the features of the present invention. The foregoing and other objects, features, aspects and advantages of the present invention will become more apparent from the following detailed description of the present invention when taken in conjunction with the accompanying drawings.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a block diagram illustrating a structure of a disease diagnosis system and a configuration of a disease diagnosing apparatus constituting the same according to an embodiment of the present invention;

FIG. 2 is a block diagram illustrating a configuration of a patch within the disease diagnosing apparatus and a diagnostic module detachably attached to the patch according to an embodiment of the present invention;

FIG. 3 is a side view illustrating a detailed configuration of the diagnostic module detachably attached to the patch within the disease diagnosis apparatus according to an embodiment of the present invention; and

FIG. 4 is a view illustrating a method for separating the diagnostic module from a diagnostic module detaching recess through a detaching button of the patch within the disease diagnosis apparatus according to an embodiment of the present invention.

### DETAILED DESCRIPTION OF THE PREFERRED EMBODIMENT

Embodiments of the present invention will now be described in detail with reference to the accompanying drawings. The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like components.

Hereinafter, a diagnostic module for simply diagnosing a disease at low cost in domestic settings, and a patch type disease diagnosis apparatus including the same will be described. In particular, a disease diagnosis apparatus for detachably attaching a plurality of diagnostic modules to a single patch and diagnosing a disease will be described. Since a plurality of diagnostic modules are detachably attached to a single patch, a patch can be re-used, having advantages in that a disease can be diagnosed efficiently at low cost, relative to an existing disease diagnosis apparatus using a single-use patch. Also, a diagnostic module integrating functions such as blood collecting, current signal detection, current signal amplifying and filtering, and the like, according to an embodiment of the present invention will be described. Through the diagnostic module, a user can simply diagnose a disease by simply applying pressure to the diagnostic module. Also, a blood collecting unit for collecting blood, a sensor unit for detecting a current signal, and a circuit for amplifying and filtering a current signal are incorporated into a single module, minimizing a distance between the sensor unit and the circuit and thus minimizing an error rate between a signal output from the sensor and a signal received by the circuit.

Hereinafter, an acute myocardial infarction index will be described as an example of a disease diagnosed by using the disease diagnosing apparatus, but the present invention may be applicable to all diseases able to be diagnosed using blood.

Also, in the following description, a mobile terminal is a terminal performing wireless communications with the disease diagnosis apparatus and may include a cellular phone, a personal communications system (PCS), a personal data assistant (PDA), an IMT-2000 (international mobile telecommunication-2000)-compliant device, a smartphone, a notebook computer, a tablet personal computer (tablet PC), and the like, for example.

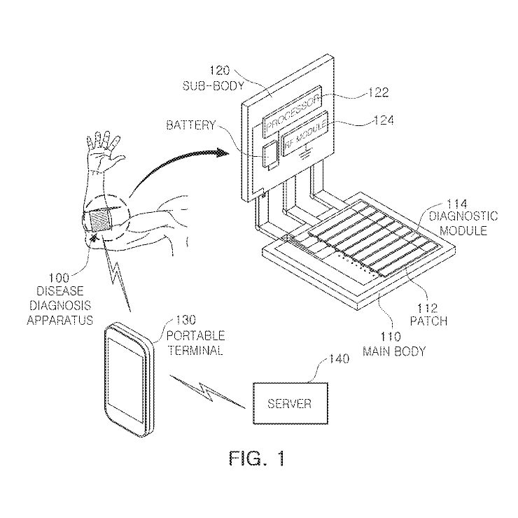

FIG. 1 is a block diagram illustrating a structure of a disease diagnosis system and a configuration of a disease diagnosing apparatus constituting the same, according to an embodiment of the present invention.

Referring to FIG. 1, the disease diagnosis system includes a disease diagnosis apparatus 100, a portable terminal 130, and a server 140.

The disease diagnosis apparatus 100 is attached to a human (or a user), automatically collects and analyzes blood from the human (or the user) based on pressure applied to the diagnostic module 114, and transmits analysis information including analysis results to a medical attendant (or doctor) by way of the portable terminal 130 and the server 140. Accordingly, when the medical attendant diagnoses a disease, the disease diagnosis apparatus 100 receives information regarding the diagnosis results by way of the server 140 and the portable terminal 130 and displays the received information on a screen.

The portable terminal 130 transmits and receives data to and from the disease diagnosis apparatus 100 and the server 140.

The server 140 transmits analysis information received from the disease diagnosis apparatus 100 by way of the portable terminal 130 to client software of the medical attendant, and transmits information regarding diagnosis results received from the client software to the disease diagnosis apparatus 100 by way of the portable terminal 130. Here, the medical attendant may diagnose whether the user has acute myocardial infarction based on the information received via the client software, and provide information regarding the diagnosis results to the disease diagnosis apparatus 100 by way of the server 140 and the portable terminal 130 through the client software.

Here, the disease diagnosis apparatus 100 may have a size of 110 mm×60 mm×10 mm, for example, and may be attached to a human body (e.g., the arm) by using a band, or the like. The disease diagnosis apparatus 100 include a main body 110 including a patch 112 and a diagnostic module 114, and a sub-body 120 having a display unit (not shown) and a plurality of (e.g., three) buttons (not shown). Here, the sub-body 120 is hinge-coupled to the main body 110 such that it may be opened and closed with respect to the main body 110.

The patch 112 provided in the main body 110 includes one or more (e.g., nine) diagnostic module attachable-detachable recesses and attachable-detachable buttons corresponding to the diagnostic module attachable-detachable recesses, respectively. The one or more diagnostic modules 114 may be detachably attached to the diagnostic module attachable-detachable recesses, respectively, automatically collect and analyze blood based on pressure applied to a corresponding diagnostic module 114, and provide analysis information to the processor 122 through an individual internal electric wire. Here, the reason why each diagnostic module 114 uses an individual electric wire in providing analysis information is to prevent a current remaining after being generated in the already used diagnostic module from interfering with a current generated in the diagnostic module 114. The used diagnostic module 114 may be separated from the diagnostic module attachable-detachable recess through the attachable-detachable button pressed by the user, and a new diagnostic module may be inserted into the corresponding diagnostic module attachable-detachable recess and used for diagnosis.

The display unit (not shown) provided in the sub-body 120 displays information regarding the diagnosis results on a screen under the control of an internal process 122, and a plurality of buttons (not shown) provide button input data corresponding to a button pressed by the user to the internal processor 122. Accordingly, the internal processor 122 may change a form of information displayed on the display unit (not shown) based on the button input data provided from the plurality of buttons (not shown). For example, the plurality of buttons (not shown) may include a first button for displaying information in a graph form on the display unit (not shown) and a second button for displaying information in a text form on the display unit (not shown). The plurality of buttons (not shown) may further include a third button for calling a medical attendant.

The interior of the sub-body 120 includes the processor 122 and a radio frequency (RF) module 124. The processor 122 may be implemented as an advanced RISC machines (ARM) processor, and process an unprocessed signal provided from the diagnostic module 114. To this end, the processor 122 may include an analog-to-digital converter (ADC), and the ADC may convert an analog signal from the diagnostic module 114 into a digital signal. The RF module 124 wirelessly transmits a processed signal from the processor 122 to the portable terminal 130 in real time, and thereafter, when information regarding diagnosis results is received through the portable terminal 130, the RF module 124 provides the received information regarding the diagnosis results to the processor 122. The processor 122 outputs the information regarding the diagnosis results to the screen through the display unit (not shown). If near-field communications (e.g., Bluetooth™) are available between the disease diagnosis apparatus 100 and the portable terminal 130, signals may be transmitted and received between the disease diagnosis apparatus 100 and the portable terminal 130 by the near-field communications.

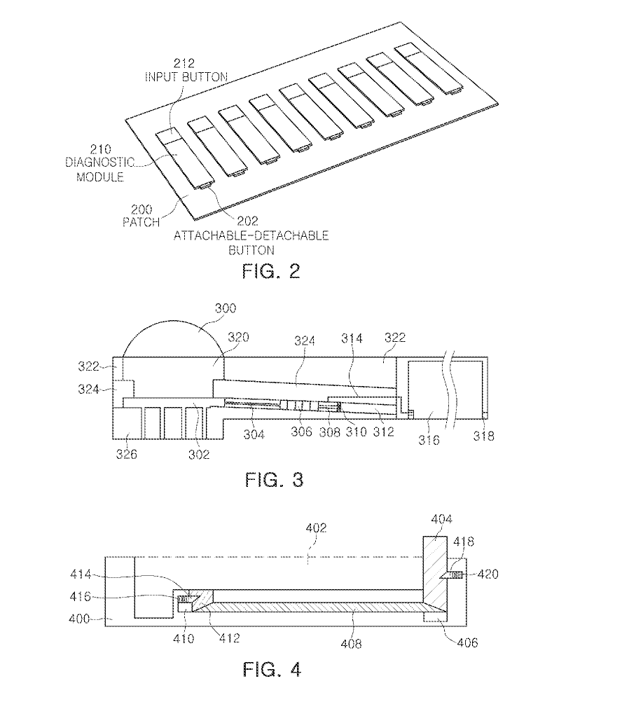

FIG. 2 is a block diagram illustrating a configuration of a patch within the disease diagnosing apparatus and a diagnostic module detachably attached to the patch according to an embodiment of the present invention.

Referring to FIG. 2, one or more diagnostic modules 210 may be detachably attached to a single patch 200. Each diagnostic module 210 automatically collects and analyzes blood based on pressure applied thereto through input button 212 and provides analysis information to the processor of the disease diagnosis apparatus through an individual electric wire. An already used diagnostic module 210 may be separated from the diagnostic module attachable-detachable recesses of the patch 200 through the attachable-detachable button 202 pressed by the user, and a new diagnostic module may be inserted into the diagnostic module attachable-detachable recess of the corresponding patch 200 so as to be used for diagnosis. Accordingly, by attaching a single patch, diagnosis can be conducted repeatedly by a desired number of times regardless of the amount of the diagnostic module attachable-detachable recesses.

FIG. 3 is a side view illustrating a detailed configuration of the diagnostic module detachably attached to the patch within the disease diagnosis apparatus according to an embodiment of the present invention.

Referring to FIG. 3, the diagnostic module includes a blood collecting unit, a sensor unit, and a high-sensitivity signal sensing circuit 316.

The blood collecting unit includes a microneedle 302 and a microfluidic chip 304. The microneedle 302 collects blood based on pressure applied to the diagnostic module through the input button 300, and in this case, the microneedle 302 collects a minimum amount of blood (e.g., 10 μl or less) required for diagnosing a disease through a minimally invasive method with respect to skin. Here, the skin may be infected in the case that the microneedle 302 is made of a metal, so in order to prevent bacterial infections, the microneedle 302 may be coated by using a parylene polymer. The microfluidic chip 304 transfers the collected blood to the sensor unit without using power (on a non-power basis).

The sensor unit includes a flow-through hole (FTH) multilayer thin film 306 and a three-dimensional (3D) electrochemical sensor 308. The FTH multilayer thin film 306 removes impurities from the blood provided from the blood collecting unit and provides a substrate so that an antigen-antibody reaction may occur within the 3D electrochemical sensor 308. The 3D electrochemical sensor 308 detects a current signal generated by an oxidation-reduction reaction between antibodies reacting to a corresponding cardiac marker and antigens in blood without impurities by cardiac markers, and provides the detected current signal to the high-sensitivity signal sensing circuit 316 through the internal electric wire 314. Here, strength of the detected current signal indicates concentration of one or more cardiac markers in the blood without impurities. After the oxidation-reduction reaction, residual elements are introduced to a waste chamber 312 through a film 310.

The 3D electrochemical sensor 308 includes an electrode array having a 3D structure, and detects a current signal generated by an oxidation-reduction reaction between specific antibodies fixed to an electrode and antigens in blood by combining an antibody immobilization technique and a specific antibody technique. The antibody immobilization technique is a technique of immobilizing an antibody with an electrode, and the specific antibody technique refers to a technique enabling an antibody to have a specific reaction to only a specific antigen. For example, in order to measure a concentration of a plurality of cardiac markers, a specific antibody reacting to each cardiac marker may be immobilized to a predetermined position of an electrode. In the case of a symptom of acute myocardial infarction (AMI), the cardiac marker includes myglobin, creatine kinase-myocardial band (CK-MB), troponin T, troponin I, and the like, generated in blood, and the 3D electrochemical sensor 308 may detect a current signal generated by an oxidation-reduction reaction between antibodies reacting to each of a plurality of (four) cardiac markers and antigens in blood without impurities.

Meanwhile, strength of the current signal detected by the 3D electrochemical sensor 308 is very weak, so it may be distorted due to ambient noise. Thus, in an embodiment of the present invention, the high-sensitivity signal sensing circuit 316 for amplifying and filtering the detected current signal is provided in the diagnostic module, and an error rate between a signal output from the 3D electrochemical sensor 308 for detecting a current signal and a signal received by the high-sensitivity signal sensing circuit 316 for amplifying and filtering a current signal can be minimized by minimizing a distance therebetween.

The high-sensitivity signal sensing circuit 316 includes an amplifying unit and a filter, and amplifies and filters the current signal detected by the 3D electrochemical sensor 308. The amplifying unit amplifies a pA-class micro-current detected by the 3D electrochemical sensor 308 and the filter filters the amplified current signal in order to protect it from noise. A connector 318 provides the amplified and filtered current signal to the internal process of the disease diagnosis apparatus through an individual electric wire. Thus, the processor processes the amplified and filtered current signal, namely, converts and digitizes the amplified and filtered current signal into a digital signal, and wirelessly transmits the processed signal to the portable terminal 130 through the RF module 124 in real time.

Meanwhile, the input button 300 is made of a soft material, and in order to allow force to be evenly applied to the entirety of the microneedle 302 when pressure is applied to the input button 300 by the user's finger, a carrier 320 made of a hard material exists between the input button 300 and the microneedle 302. A first frame 322 is made of a hard material to fix the carrier 320, and a second frame 324 is made of a soft material to allow the carrier 320 to apply pressure to the microneedle 302 according to pressure applied to the input button 300. A third frame 326 is made of a soft material allow the microneedle 302 to be injected into skin according to pressure applied to the input button 300, and in order to minimize interference of a human body, a nonconductive material may be used. Here, degrees of hardness of the second frame 324 and the third frame 326 are lower than that of those of the first frame 322 and the carrier 320.

FIG. 4 is a view illustrating a method for separating the diagnostic module from a diagnostic module attachable-detachable recess through an attachable-detachable button of the patch within the disease diagnosis apparatus according to an embodiment of the present invention.

Referring to FIG. 4, the patch 400 includes a diagnostic module attachable-detachable recess 402 as an empty space in which a diagnostic module is inserted and a rod-type attachable-detachable button 404 pressed by the user in order to separate a diagnostic module from the diagnostic module attachable-detachable recess 402. In a state in which a diagnostic module is inserted in the diagnostic module attachable-detachable recess 402, when the user presses the attachable-detachable button 404, the attachable-detachable button 404 is lowered to the bottom of a first space 406, automatically pushing a first rod 408 leftwardly. Accordingly, the first rod 408 is pushed down to the end of a second space 410, pushing a second rod 412 upwardly. Accordingly, the second rod 412 automatically pushes the diagnostic module insertedly positioned in the diagnostic module attachable-detachable recess 402 in an upwardly, and thus, the diagnostic module can be separated from the diagnostic module attachable-detachable recess 402. A third rod 414 and a fourth rod 418 serve to fix the second rod 412 and the attachable-detachable button 404 such that they may not move outside of the patch 400. A first spring 416 and a second spring 420 serve to provide a shove to the third rod 414 and the fourth rod 418 to constantly fix the second rod 412 and the attachable-detachable button 404.

As set forth above, according to embodiments of the invention, the diagnostic module capable of simply diagnosing at low cost in household and a disease diagnosis apparatus including the same can be provided.

While the present invention has been shown and described in connection with the embodiments, it will be apparent to those skilled in the art that modifications and variations can be made without departing from the spirit and scope of the invention as defined by the appended claims.

What is claimed is:

1. A disease diagnosis apparatus comprising:
a patch including one or more diagnostic module attachable-detachable recesses;
one or more diagnostic modules detachably attached to the diagnostic module attachable-detachable recesses to collect and analyze blood; and
a processor processing analysis results.

2. The disease diagnosis apparatus of claim 1, wherein the patch further includes attachable-detachable buttons corresponding to the diagnostic module attachable-detachable recesses, respectively,
wherein each of the diagnostic modules is separated from the diagnostic module attachable-detachable recesses through an attachable-detachable button pressed by a user.

3. The disease diagnosis apparatus of claim 1, wherein each of the diagnostic modules comprises:
a blood collecting unit collecting blood using a microneedle, based on pressure applied to a corresponding diagnostic module;
a sensor unit detecting a current signal generated by an oxidation-reduction reaction between antibodies reacting to a corresponding cardiac marker, among cardiac markers, and antigens contained in the collected blood by using a three-dimensional (3D) electrochemical sensor; and
a high-sensitivity signal sensing circuit amplifying and filtering the detected current signal and providing the amplified and filtered current signal to the processor,
wherein the blood collecting unit, the sensor unit, and the high-sensitivity signal sensing circuit are integrated into a single module.

4. A diagnostic module comprising:
a blood collecting unit collecting blood using a microneedle, based on pressure applied to a corresponding diagnostic module;
a sensor unit detecting a current signal generated by an oxidation-reduction reaction between antibodies reacting to a corresponding cardiac marker, among cardiac markers, and antigens contained in the collected blood by using a three-dimensional (3D) electrochemical sensor; and
a high-sensitivity signal sensing circuit amplifying and filtering the detected current signal,
wherein the blood collecting unit, the sensor unit, and the high-sensitivity signal sensing circuit are integrated into a single module.
